(19) United States
(12) Patent Application Publication
(10) Pub. No.: US 2007/0298422 A1
(43) Pub. Date: Dec. 27, 2007

(54) Procedure for the analysis of a sample

(76) Inventors: Jorn Mosner, Erlangen (DE); Sebastian Schmidt, Weisendorf (DE)

Correspondence Address: HARNESS, DICKEY & PIERCE, P.L.C., P.O.BOX 8910, RESTON, VA, 20195, US

(21) Appl. No.: 11/656,463

(22) Filed: Jan. 23, 2007

(30) Foreign Application Priority Data
Jan. 24, 2006 (DE) 10 2006 003 415.5

(51) Int. Cl. C12Q 1/68

(52) U.S. Cl. 435/6

(57) ABSTRACT

A procedure for the analysis of a sample is disclosed, where the sample includes at least one nucleic acid molecule having at least one nucleic acid sequence. In at least one embodiment, the procedure includes making available a comparison molecule of known concentration having a known comparison sequence, where the comparison sequence has a defined mass difference in comparison to a reference sequence of the nucleic acid sequence; cleaving the nucleic acid sequence to give fragments of the nucleic acid sequence and the comparison sequence to give fragments of the comparison sequence, determining a mass spectrum of a mixture of the fragments of the nucleic acid sequence and of the comparison sequence by means of mass spectrometry, determining a comparison spectrum of the fragments of the comparison sequence by means of mass spectrometry, determination of the concentration of the nucleic acid sequence from the mass spectrum and the comparison spectrum and determining still unknown sequence variations of the nucleic acid sequence in comparison to the reference sequence from the mass spectrum and a reference spectrum of the reference sequence.

### PRIORITY STATEMENT

The present application hereby claims priority under 35 U.S.C. §119 on German patent application number DE 10 2006 003 415.5 filed Jan. 24, 2006, the entire contents of which is hereby incorporated herein by reference.

### FIELD

Embodiments of the present invention generally relates to a procedure for the analysis of a sample, for example one where the sample comprises at least one nucleic acid molecule having at least one nucleic acid sequence.

### BACKGROUND

The importance of molecular biological analyses is gaining increasing importance in medicine. For example, pathogens in the body of a patient can thus be detected and quantified using appropriate methods. Here, the pathogen is identified, for example, by means of characteristic DNA sequences, such that an appropriate treatment for the patient can be worked out. In this regard, the determination of DNA sequences and their detection is of increasing importance.

At present, DNA sequencing is carried out either by the chemical degradation procedure of Maxam and Gilbert (Methods in Enzymology 65, 499-560 (1980)) or by the enzymatic dideoxy-nucleotide termination procedure of Sanger et al. (Proc. Natl. Acad. Sci. USA 74, 5463-67 (1977)). In the chemical procedure, base-specific modifications lead to a base-specific cleavage of the radioactive- or fluorescent-labeled DNA fragment. Using the four separate base-specific cleavage reactions, four sets of nested fragments are prepared, which are separated according to length by polyacrylamide gel electrophoresis (PAGE). After autoradiography, the sequence can be directly read, since each band (fragment) in the gel results from a base-specific cleavage event. The fragment lengths of the four “ladders” are therefore translated directly into a specific position in the DNA sequence.

Gel electrophoresis has some disadvantages. The preparation of a homogeneous gel by polymerization, loading of the samples, the electrophoresis itself, the detection of the sequencing pattern (e.g. by autoradiography), removal of the gel and cleaning of the glass plates in order to prepare another gel are very labor-intensive and time-consuming processes. Moreover, the whole process is error-prone, difficult to automate, and trained and experienced personnel are needed in order to improve the reproducibility and reliability. In the case of radiolabeling, the autoradiography itself can last from hours to days. In the case of fluorescent labeling, at least the detection of the sequencing bands can be carried out automatically if devices for laser scanning are used which are integrated into commercially available DNA sequencers. One problem which is associated with fluorescent labeling is the influence of the four different base-specific fluorescent tags on the mobility of the fragments during the electrophoresis and a possible overlapping in the spectral bandwidth of the four specific dyes, which reduces the distinguishing power between adjacent bands and thus increases the probability of sequence ambiguities.

In general, mass spectrometry makes available a device/method for the “weighing” of individual molecules, in that the molecules are ionized in a vacuum and made to “fly” by vaporization. Under the influence of combinations of electrical and magnetic fields, the ions follow flight paths which depend on their individual mass and charge. In the field of molecules of low molecular mass, mass spectrometry has for a long time been part of the routine physical-organic repertoire for the analysis and characterization of organic molecules by the determination of the mass of the parent molecular ion.

Additionally, by collisions of this parent molecular ion being caused with other particles (e.g. argon atoms), the molecular ion is fragmented by “collision-induced dissociation” (CID), secondary ions being formed. The fragmentation pattern or the fragmentation route often makes it possible to derive detailed structural information. On account of the apparent analytical advantages of mass spectrometry in the provision of high detection sensitivity, the accuracy of the mass measurements and the speed, as well as online data transfer to a computer, there is considerable interest in the use of mass spectrometry for the analysis of nucleic acids.

Mass spectrometry as a method of measuring the size of the amplified DNA segments has considerable advantages: it is not the ionic mobility in liquid or polymerized gel which is measured, but directly the mass of the ions. The ionic mobility depends critically on form factors and it can therefore occur that the (sometimes randomly assumed) folding structure of the molecules results in a corruption of the measurements. However, the (sometimes randomly differing) degree of polymerization of the gel also influences the migration rate of the DNA segments, such that artifacts frequently occur.

These types of artifacts are not to be expected in mass spectrometry. In principle, mass spectrometry moreover offers a higher accuracy of the mass determination. However, even the mass spectrometric measurement of the masses of the DNA segments is subject to restrictions. The measurement of the DNA generally and in particular the accuracy of the mass determination is particularly influenced by three effects: (1) adduct formation by ubiquitous Na or K ions, which accumulate in random amounts on the DNA segments, (2) the formation of double peaks by different masses of the two DNA single strands and (3) the fragmentation of the ssDNA segments to be measured during the MALDI process, usually by cleavage of the bases during the ionization. All three effects lead to a blurring of the peaks: adduct formation to higher masses, fragmentation to lower masses, and double peak formation to spreading often no longer recognizable as a double peak. In the high mass range, these effects act such that measurements can no longer be carried out accurately on a nucleotide.

Mass spectrometry procedures are, for example, electro-spray/ion spray (ESI), ion-cyclotron resonance (ICR) and matrix-assisted laser desorption/ionization (MALDI). MALDI mass spectrometry can be improved if a time-of-flight (TOF) configuration is used as a mass analyzer. The present rapid progress in the MALDI technique leads to a high degree of automation of the sample ionization and to short analysis times per sample. Molecular weight determinations of even very large analytical molecules in amounts of a few hundred attomol in measuring periods of a few seconds are thus possible. Hundreds of samples can be accommodated on a sample carrier. Using mass spectrometric analysis, automation and the high density of samples open up the possibility of massive parallel processing of some ten thousand samples per day.

MALDI is an ionization procedure for large molecular analyte substances on surfaces. The analyte substances are transferred to a surface together with suitable matrix substances of a relatively small molecular weight in solution relative to the analyte molecules, dried there and irradiated with a laser pulse of a few nanoseconds duration. A small amount of matrix substance vaporizes, a few molecules thereof as ions. The very dilute analyte substance in the matrix, whose molecules are isolated on dilution, is vaporized here, even if its vapor pressure is normally not adequate for vaporization. The relatively small ions of the matrix substance react with the large molecules of the analyte substance with the result that the analyte substance molecules remain as ions due to proton transfer. Double-stranded DNA (dsDNA) is reduced here to single stranded DNA (ssDNA) in the acidic matrix.

Procedures for the sequencing of the DNA according to the Sanger scheme utilizing PCR procedures and mass spectrometers using the MALDI or ESI technique are known, for this see EP 0 679 196 B1.

From U.S. Pat. No. 6,238,871 B1, a procedure is known by which a nucleic acid sequence can be determined. In this, the nucleic acid sequence is cleaved base-specifically such that base-specifically ending fragments result. The fragments are investigated by way of mass spectrometry, by which a mass spectrum having different maxima corresponding to the fragments results. By analysis of the mass spectrum, the sequence of the nucleic acid can be determined. Here, the fragment length and the molecular weight connected with it are taken into account. The method described is based on the Sanger sequencing method and offers the advantage that no gel electrophoresis or similarly laborious procedures are necessary.

In EP 0 815 261 B1, it is described how DNA sequences can be detected by means of mass spectrometry. For this, detector oligonucleotides having one or more nucleic acid molecules are hybridized. Subsequently, ionization and vaporization of the hybridized product takes place. The ionized and vaporized nucleic acid is analyzed by way of mass spectrometry, in which the detection of the detector oligonucleotide by mass spectrometry indicates the presence of the target nucleic acid sequence in the sample. In general, before the hybridization the nucleic acid molecule is amplified using known amplification procedures, such as, for example, the polymerase chain reaction (PCR). For the determination of the mass spectrum, various procedures can be used. Examples of these are time-of-flight mass spectrometry, matrix-assisted laser desorption/ionization and electron spray procedures. If a number of target nucleic acid sequences are detected, various detector oligonucleotides having various masses are hybridized to the target nucleic acid sequences. In this way, the various target nucleic acid sequences can be detected in the mass spectrum.

PCR is the targeted amplification of an accurately selected piece of the two-stranded DNA. The selection of the DNA segment is carried out by means of a pair of “primers”, two ssDNA fragments each having a length of approximately 20 bases, which (described in a somewhat abbreviated and simplified manner) are hybridized on both sides (the future ends) of the selected DNA fragment. The amplification is carried out in a single temperature cycle by an enzyme by the name of polymerase, which represents a chemical factory in a molecule. The PCR reaction proceeds in aqueous solution, in which a few molecules of the starting DNA to be amplified and sufficient amounts of polymerase, primers, nucleic acids and stabilizers are present. In each temperature cycle (for example melting of the double helix at 92° C., hybridization of the primers at 55° C., reintegration to give a double helix by addition of new DNA members by the polymerase at 72° C.), the selected DNA segment is in principle doubled. In 30 cycles, around a billion DNA segments are thus produced from a single double strand of the DNA as starting material. (Strictly described, the two primers hybridize to the two different single strands of the DNA and the shortening on the selected DNA segment from one primer to the other occurs only statistically on further amplification).

Under optimum conditions, the polymerase can add approximately 500 to 1000 bases per second to the primer. With an adequate excess of primers, polymerase and substrate, the cycle time depends virtually only on the heating and cooling rate, which in turn depends on the liquid volume, vessel volume, heat conductivity, inter alia. In principle, each temperature stage only has to be maintained for a few seconds.

The primers are part of the amplified DNA segments, in this way they are exhausted during the PCR amplification. This offers the advantage that, for example, chemical groups (such as, for example, fluorescent or particularly well-ionizing groups) can be incorporated, which can be utilized for later detection. The chemical groups are added beforehand to the synthetically prepared primers.

By way of the addition of only one pair of primers, uniform DNA segments can be amplified. If, on the other hand, a number of different pairs of primers are to be added simultaneously, a number of uniform DNA segments are also amplified simultaneously (“multiplexed PCR”). This type of multiplexed PCR is frequently used and often has particular advantages. For “fingerprinting” for the identification of individuals by means of DNA segments of variable length (“VNTR=Variable Number of Tandem Repeats” or “AMP-FLP=Amplified Fragment Length Polymorphism”), it makes the analyses more rapid. Here, it can be achieved by the choice of the primer, whose spacing determines the mean molecular weight of the DNA segments, that the variations in the molecular weight of the DNA segments formed by the different primer pairs do not or only rarely overlap. This type of multiplex PCR requires an analyzer which is capable of the simultaneous measurement of a large range of molecular weights.

The measurement methods mentioned can be employed, for example, in the therapy of chronic infections, such as, for example, with the HI virus. Here, for the checking of the therapeutic success and course, the concentration of the viruses in the blood of a patient, the “virus load” is regularly determined. If an increase in the virus load is found in the analysis in comparison to preceding investigations, the development of resistance of the virus to the medicaments used in the therapy can be concluded. Correspondingly, in the case of a finding of this type the medication is to be changed accordingly.

Resistances of the virus are in general based on a mutation in the DNA of the virus. For the effective adjustment of the medication, it is therefore necessary to sequence the DNA of the virus. The determined sequence of the gene is compared with known sequences such that conclusions can be made on mutations or sequence variations which lead to medicament resistance.

A customary procedure for the determination of the virus load per blood volume consists in amplifying the virus gene and a comparison gene simultaneously and subsequently detecting them by means of optical detection. From the detection, a concentration ratio between the virus gene and the comparison gene is derived, such that the concentration of the virus gene can be deduced at a known concentration of the comparison gene. This described principle is used, for example, in the system “Amplicor©” of Hoffmann-La Roche.

For the determination of the virus load by way of the mass spectrometry procedure already described, the virus gene to be analyzed is first jointly amplified with a comparison gene, for example by way of PCR or “primer extension”. By means of primer extension, short nucleic acid fragments are then produced whose mass is different for the virus gene and the comparison gene. After determination of the mass spectrum, the concentration ratio of virus gene and comparison gene can be determined by way of the masses of the virus and of the comparison gene, which have been chosen to be different. By addition of the known concentration of the comparison gene, the concentration of the virus gene and thus of the virus in the sample can be determined.

The determination of the gene sequence of the virus gene can be carried out, for example, by sequencing according to Sanger. This is the case, for example, in the system “Trugene©” of Bayer, in which gel electrophoresis is used for sequencing.

For sequencing by way of mass spectrometry, the virus gene is first in general amplified, where already modified bases which facilitate a specific cleavage can be incorporated. The amplified virus gene is cleaved specifically at certain bases or sequence motifs, for example by addition of appropriate restriction enzymes. The masses of the resulting fragments can be determined by means of mass spectrometry, from which conclusions on the sequence of the gene investigated are possible.

Often, both procedures, that is the determination of the virus load and the sequencing of the virus DNA, are carried out in succession for the same patient. Thus, in the case of a determined, increased virus load the continuative therapy can be defined and selectively adjusted. Both diagnoses have hitherto in general been carried out on different equipment using laborious procedures, such that high costs result here for personnel and consumable supplies. Moreover, a postponement of the diagnostic decision occurs, since the procedures are in general time-consuming. Since the resistance behavior of known virus strains becomes greater with time and the choice of therapeutics increases, in future the appropriate analyses will have increased importance.

In “Quantitative Analysis of Plasma TP53 249^{Ser}-Mutated DANN by Electrospray Ionization Mass Spectrometry”, Cancer Epidemiol Biomarkers Prev (2005) 14(12) 2956-2962, M. E. LLeonart et al. describe a procedure to determine the concentration and the occurrence of a specific mutation by means of mass spectrometry. Here, internal standards are used which, compared to a wild type, contain the sought mutation and a specific replacement base. In this way, the occurrence of the 249^{Ser} mutation and its concentration in samples can be investigated.

### SUMMARY

In at least one embodiment of the present invention, a procedure is specified for the analysis of a sample, by which both a mutation and the concentration of a nucleic acid can be determined.

According to at least one embodiment, a procedure for the analysis of a sample is specified, where the sample comprises at least one nucleic acid molecule having at least one nucleic acid sequence, comprising the following process steps:
making available a comparison molecule of known concentration having a known comparison sequence, where the comparison sequence has a defined mass difference in comparison to a reference sequence of the nucleic acid sequence,
cleaving the nucleic acid sequence to give fragments of the nucleic acid sequence and of the comparison sequence to give fragments of the comparison sequence,
determining a mass spectrum of a mixture of the fragments of the nucleic acid sequence and the comparison sequence by means of mass spectrometry,
determining a comparison spectrum of the fragments of the comparison sequence by means of mass spectrometry,
determination of the concentration of the nucleic acid sequence from the mass spectrum and the comparison spectrum and
determining still unknown sequence variations of the nucleic acid sequence in comparison to the reference sequence from the mass spectrum and a reference spectrum of the reference sequence.

The procedure described offers the advantage that in the course of an individual analysis both the concentration of the nucleic acid sequence to be determined and the determination of sequence variation in comparison to a reference sequence is possible. By way of the insertion of the comparison sequence having a defined mass difference into the nucleic acid sequence, the concentration ratio of the comparison molecule and of the nucleic acid molecule can be determined within the mass spectrum. As with known procedures, the concentration of the nucleic acid molecule can be determined here. The same mass spectrum can also be used for the determination of sequence variations by making a comparison with a reference spectrum of the reference sequence. In this way, the analyses which otherwise have to be carried out in separate procedures can be carried out in a single procedure.

The procedure for the analysis of a sample in at least one embodiment, where the sample comprises at least one nucleic acid molecule having at least one nucleic acid sequence, comprises the following process steps:
making available a comparison molecule of known concentration having a known comparison sequence, the comparison sequence having a defined mass difference in comparison to a reference sequence of the nucleic acid sequence,
simultaneous amplification of the nucleic acid molecule and of the comparison molecule,
cleavage of an amplification product to give fragments of the nucleic acid sequence and to give fragments of the comparison sequence,
determining a mass spectrum of a mixture of the fragments of the nucleic acid sequence and of the comparison sequence by means of mass spectrometry,
determining a comparison spectrum of the fragments of the comparison sequence by means of mass spectrometry,
determination of the concentration of the nucleic acid sequence from the mass spectrum and the comparison spectrum and
determining still unknown sequence variations of the nucleic acid sequence in comparison to the reference sequence from the mass spectrum and a reference spectrum of the reference sequence.

The procedure offers the same advantages as the procedure in the former embodiment, but an amplification of the nucleic acid molecule and of the comparison molecule is carried out in at least one other embodiment. This is the case, in particular, when too few copies of the nucleic acid molecule are present in the sample.

The amplification can be carried out, for example, by cloning, transcription-based amplifications, polymerase chain reactions (PCR), ligase chain reaction (LCR) or strand displacement amplification (SDA).

In an example embodiment of the invention, the comparison sequence is based on a reference sequence corresponding to the nucleic acid sequence. The mass difference is produced by replacing at least one starting base occurring in the comparison sequence by at least one labeling base. This has the advantage that the mass difference occurring in the mass spectrum clearly stands out and thus the analysis of the mass spectrum is facilitated.

In an advantageously designed procedure, the cleavage of the nucleic acid sequence and the cleavage of the comparison sequence are carried out by addition of at least one restriction enzyme (endonuclease). The use of restriction enzymes for the cleavage of nucleic acid sequences is known. There are a multiplicity of restriction enzymes available which dissect the nucleic acid at specific bases. By means of the known cleavage point, conclusions can be made on the sequence of the nucleic acid from the mass spectrum and the molecular weight determined therein.

A procedure is particularly advantageous of the type where the labeling base and the restriction enzyme are chosen such that the nucleic acid sequence is cleaved at the starting base and the comparison sequence is not cleaved at the labeling base. Thus a fragment smaller due to cleavage with the restriction enzyme than in the nucleic acid sequence results in the comparison sequence. Consequently, three maxima occur in the mass spectrum, which are unequivocally identifiable by their mass difference.

A procedure of at least one embodiment is advantageous of the type where the determination of the concentration of the nucleic acid sequence comprises the following procedure steps:
analysis of maxima occurring in the mass spectrum and corresponding to the fragments,
identification of one of the maxima occurring, which corresponds to the fragment of the comparison sequence which comprises the labeling base,
identification of two further maxima which correspond to the two fragments of the nucleic acid sequence which result due to the cleavage of the nucleic acid sequence at the starting base,
determination of a concentration ratio of the nucleic acid molecule and of the comparison molecule by determination of the area ratio of one maximum to the sum of the two other maxima and
determination of the concentration of the nucleic acid molecule by division of the concentration ratio by the concentration of the comparison molecule.

By way of the procedure extended in this way, the concentration of the nucleic acid sequence can be determined in a simple manner.

A further advantageous procedure comprises, for the determination of the sequence variations, the following process steps:
standardization of the comparison spectrum to the concentration of the comparison sequence,
determination of a difference spectrum by subtraction of the standardized comparison spectrum from the mass spectrum,
determination of the sequence variations by comparison of the difference spectrum with the reference spectrum.

Thus, the sequence variations occurring can also be determined from the same mass spectrum which has already been used for determining the concentration of the nucleic acid molecule.

### BRIEF DESCRIPTION OF THE DRAWINGS

Further advantages of the invention are clear with the aid of the example embodiment described below in connection with the attached drawings. The latter show:

FIG. 1: a schematic flow diagram of an example embodiment of the invention,

FIGS. 2 to 5: various schematic mass spectra during different process steps.

### DETAILED DESCRIPTION OF THE EXAMPLE EMBODIMENTS

It will be understood that if an element or layer is referred to as being “on”, “against”, “connected to”, or “coupled to” another element or layer, then it can be directly on, against, connected or coupled to the other element or layer, or intervening elements or layers may be present. In contrast, if an element is referred to as being “directly on”, “directly connected to”, or “directly coupled to” another element or layer, then there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

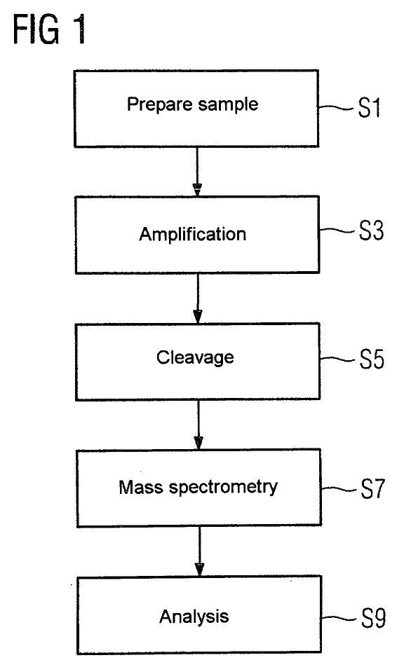

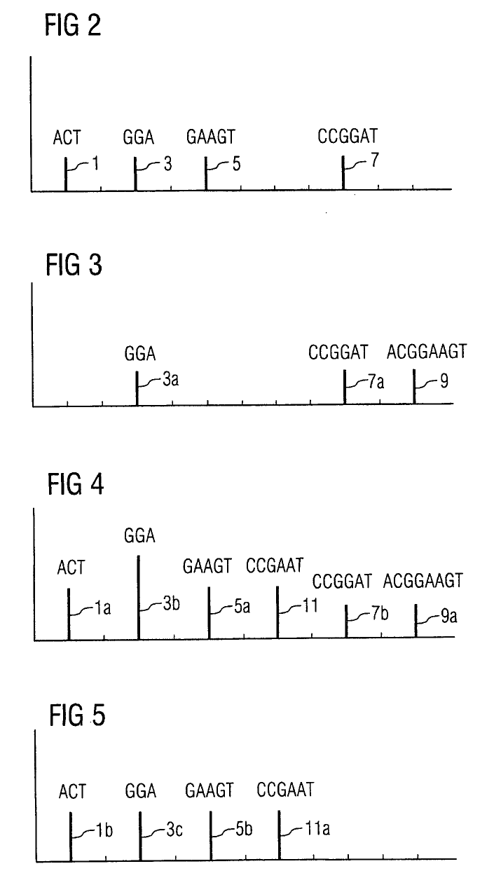

With the aid of FIG. 1, the course of the procedure for the determination of the concentration and the sequence of a DNA are first to be described. Details for the analysis of the mass spectra determined are illustrated by exemplary mass spectra with the aid of FIGS. 2 to 5. The procedure described can be employed for a multiplicity of investigations, for example in the therapy of HIV diseases or hepatitis.

According to FIG. 1, in a first process step S1 a sample is taken and prepared for analysis. For instance, a blood sample is taken from an HIV patient, from which the virus RNA of the HI virus is extracted by appropriate preparation and made available to the further procedure. It is initially necessary here to concentrate the HI viruses and to extract the virus RNA from the viruses by cell disruption. In order to be able to perform an analysis, the conversion of the virus RNA into a corresponding cDNA sequence is necessary. This can be carried out, for example, by addition of reverse transcriptase. Various procedures are known for the purification and isolation of the RNA or DNA sequence. Thus, the appropriate DNA sequences can be hybridized, for example by means of immobilized capture oligonucleotides, and purified by rinsing with a wash solution. By releasing the hybridization, the corresponding DNA sequences can be further processed.

A known comparison sequence which is nearly identical to the nucleic acid sequence to be detected is added to the DNA sequence which is purified and made available. It originates from a corresponding comparison molecule and, in comparison to the nucleic acid sequence to be detected, has a measurable mass difference. The comparison sequence is produced by replacing a starting base by a labeling base in a known reference sequence of a reference molecule which is identical to the nucleic acid molecule to be analyzed except for the still unknown sequence variations. This is illustrated in detail with the aid of FIGS. 2 to 5.

The added comparison sequence or the added comparison molecule is amplified together with the nucleic acid molecule in a second process step S3. By way of the amplification, the nucleic acid sequence and the comparison sequence simultaneously are often amplified such that they are detectable later by means of mass spectrometry. Amplification can be carried out using known procedures, such as, for example, PCR. Should sufficient copies of the nucleic acid sequence and the comparison sequence be present in the sample, the amplification can also be dispensed with.

After the amplification, in a third process step S5 a restriction enzyme is added by which the amplification product, that is the nucleic acid sequence and the comparison sequence, are cleaved at base-specific sites. The restriction enzyme is chosen here such that the comparison sequence on the labeling base is not cleaved, but the nucleic acid sequence on the starting base is cleaved.

In the choice of the starting base which is to be replaced by a labeling base in the comparison sequence, it is important to choose a part of the reference sequence which is definitely not affected by a relevant sequence variation of the virus genome. The starting base can thus be replaced by the labeling base without influencing the investigation result of the sequencing. To this end, a sequence of the virus genome is used which has low mutation rates.

The nucleic acid sequence is known, for example, from earlier investigations except for the sequence variations occurring and which are to be investigated. In comparison to the known reference sequence, it is necessary to determine the sequence variation. The comparison sequence and thus the comparison molecule now result, for example, by synthesis of the reference molecule, in which, however, the starting base is replaced selectively by the labeling base. The labeling base is chosen here such that it is definitely not affected by a sequence variation in the still unknown nucleic acid molecule.

The starting base is therefore present with high probability in the nucleic acid sequence of the nucleic acid molecule to be investigated, such that the comparison sequence differs at the positions of the sequence variations which have occurred and on the labeling base of the nucleic acid sequence. This is illustrated in detail in the description of FIG. 2.

Owing to the cleavage of the nucleic acid sequence and the comparison sequence, base-specifically ending fragments result. As a result of the choice of the labeling base and of the restriction enzyme, one fragment more than in the comparison nucleic acid results in the nucleic acid.

In a fourth process step S7, a joint mass spectrum of the cleaved nucleic acid sequence and the cleaved comparison sequence is determined. In the mass spectrum, maxima occur which correspond to the fragments of the nucleic acid sequence and of the comparison sequence produced. In a majority of the fragments, both nucleic acid sequences agree. Only the fragments in which sequence variations are present and in the fragment which was modified by the labeling base do the fragments and thus the maxima occurring differ. Since the height of the maxima occurring depends on the number of fragments of the particular type present, consequently maxima of variously high intensity occur.

The mass spectrum can be determined, for example, by time-of-flight mass spectrometry, matrix-assisted laser desorption/ionization (MALDI), electrospray ionization (ESI), ion cyclotron resonance (ICR) or a combination of these procedures.

Additionally, a mass spectrum of the comparison sequence is determined in the fourth process step. This can be carried out either by a separate measurement of a mass spectrum or by a simulation based on the known comparison sequence.

In a fifth process step S9, the analysis of the mass spectrum determined and the determination of the concentration of the nucleic acid molecule and thus of the pathogen, and the determination of sequence variations of the nucleic acid sequence in comparison to the reference and to the comparison sequence are carried out. The analysis is illustrated in detail with the aid of FIGS. 2 to 5.

In FIGS. 2 to 5, various mass spectra of the cleaved nucleic acids involved are shown schematically. Here, the positions of the relevant maxima are indicated only by lines, the curve actually measured not being shown for reasons of clarity. On the horizontal axis in FIGS. 2 to 5, a scale of increasing mass is plotted, while in the vertical direction the intensity of the measured spectrum is plotted.

In the present example embodiment, it is assumed that the known reference sequence has the following sequence:

ACTGAAGTCCGGATGGA

Here, the letters A, T, G and C stand for the four bases adenine, thymine, guanine and cytosine, from which nucleic acid sequences are constructed. The reference sequence is, for example, known from a preceding sequencing. The comparison sequence used is the following sequence:

ACGGAAGTCCGGATGGA.

The comparison sequence differs from the reference sequence only in the third base, where in the reference sequence thymine is present as the starting base, while in the comparison sequence guanine is present as the labeling base. The corresponding fragments of the comparison sequence and the reference sequence thus have a detectable mass difference. The nucleic acid, which is still unknown and to be analyzed in parts, should have the following sequence in the present exemplary embodiment:

ACTGAAGTCCGAATGGA.

The unknown nucleic acid sequence differs only in a mutation reference sequence, consequently an individual sequence variation is present. A guanine base was replaced here by the adenine base shown emphasized. It is aimed at determining this mutation by the embodiment of present procedure. Simultaneously, it is to be determined at what concentration the nucleic acid is present in the blood sample taken from the patient. It is to be taken into account that the base thymine in the third position is present both in the reference sequence and in the nucleic acid sequence.

The nucleic acid sequences present are dissected base-specifically into individual fragments behind the thymine by the restriction enzyme used. Thus, the reference nucleic acid is dissected, for example, behind the third, the eighth and the fourteenth base into the following four fragments.

ACT GAAGT CCGGAT GGA

In a corresponding mass spectrum of the reference nucleic acid, which is shown in FIG. 2, consequently four maxima 1, 3, 5 and 7 occur. The maximum 1 corresponds to the fragment ACT, the maximum 3 to the fragment GGA, the maximum 5 to the fragment GAAGT and the maximum 7 to the fragment CCGGAT.

The comparison sequence, on the other hand, is cleaved only on the eighth and the fourteenth base, such that only the three following fragments are present:

ACGGAAGT CCGGAT GGA

Consequently, also in the corresponding mass spectrum which is shown schematically in FIG. 3, only three maxima 3a, 7a and 9 occur. The two maxima 3a and 7a correspond to the maxima 3 and 7 of the mass spectrum of the reference molecule shown in FIG. 2 and thus to the fragments GGA and CCGGAT. The maxima 1 and 5 shown in FIG. 2 do not occur in the mass spectrum of the comparison molecule, since these two fragments were not cleaved on account of the replaced labeling base. Instead of this, a new maximum 9 occurs, which corresponds to the fragment ACGGAAGT and has a significantly higher mass value.

The nucleic acid molecule is cleaved into the following fragments by the restriction enzyme:

ACT GAAGT CCGAAT GGA

Except for the mutation, these correspond to the fragments of the reference molecule.

In FIG. 4, the mass spectrum of the mixture of the nucleic acid sequence and the comparison sequence determined in the fourth process step S7 is shown schematically. Corresponding to the fragments occurring in the mixture, maxima appear within the mass spectrum. A maximum 1a (ACT) appears, which corresponds to maximum 1 of the mass spectrum shown in FIG. 2. It corresponds to the fragment which was divided off on the starting base. The fragment 3b (GGA) occurring corresponds to the fragment divided off on the fourteenth base and likewise occurs as maximum 3 in FIG. 2 and as maximum 3a in FIG. 3.

The maximum 5a occurring corresponds to the maximum 5 in FIG. 2 and thus to the fragment GAATT divided off before the eighth base. This fragment did not occur in FIG. 3, since the comparison molecule was not cleaved on the labeling base of the restriction enzyme. In addition, the maxima 7b (CCGGAT) and 9a (ACGGAAGT) occur which correspond to the maxima 7a and 9 from FIG. 3. The maximum 9a corresponds to the fragment of the labeling sequence not divided by the labeling base. This does not occur in the nucleic acid sequence, such that only the fragments of the comparison molecule contribute to the maximum 9a.

In the maxima 1a and 5a present, only the fragments of the nucleic acid sequence contribute to the intensity of the maximum, since they do not occur in the comparison sequence. Both the nucleic acid sequence and the comparison sequence contribute to the intensity of the maximum 3b, since the corresponding fragment occurs in both nucleic acid sequences. On account of the sequence variations present in the comparison between the nucleic acid molecule and the comparison molecule, two maxima 7b occur in the spectrum. The maximum 7b and 11 corresponds to the maximum 7a from FIG. 3 and thus to the fragment CCGGAT of the reference molecule not affected by the sequence variation. As a result of the sequence variation, a new maximum 11 occurs in the spectrum, which corresponds to the fragment CCGAAT.

For the determination of the concentration of the nucleic acid molecule, the maxima 1a, 5a and 9a are used for the analysis. It is known of these maxima to which fragments they correspond, since here the starting base was replaced selectively by the labeling base. The areas not shown here under the maxima 1a and 5a correspond in the sum of the relative concentration of the nucleic acid molecule. However, the absolute concentration cannot be determined without problem from the area, since a reference is lacking. As a reference, the area under the maximum 9a is used, which corresponds to the relative concentration of the comparison molecule.

Since the comparison molecule was added in a defined concentration, the absolute value for the concentration of the comparison molecule is used for standardization of the areas under the maxima. The absolute concentration of the nucleic acid molecule can thus be determined. This is carried out by summation of the areas under the maxima 1 a and 5a with subsequent division by the area of the maximum 9a. Subsequently, the result is multiplied by the known concentration of the comparison molecule, from which the concentration of the nucleic acid molecule results.

For the determination of the sequence variation, the mass spectrum of the comparison sequence shown in FIG. 3 is standardized to the height of the maximum 7b or 9a in FIG. 4. The comparison spectrum shown can be determined by a measurement or a simulation. Subsequently, the standardized spectrum of the mass spectrum shown in FIG. 4 is subtracted, such that only a mass spectrum of the nucleic acid sequence to be determined remains. By way of the preceding standardization, the maxima of the comparison sequence are completely removed.

A corresponding mass spectrum is shown schematically in FIG. 5. Here, only the maxima 1b (ACT), 3c (GGA), 5b (GAAGT) and 11a (CCGAAT) occur. By comparison with the mass spectrum of the reference molecule from FIG. 2, the sequence variation can be determined. It is found that instead of the original maximum 7 (CCGGAT) the shifted maximum 11a (CCGAAT) occurs. With the aid of the absolute mass shift between the spectrum and the knowledge of the sequence of the fragment CCGGAT of the reference molecule, it can be determined which sequence variation is present.

Consequently, the concentration, for example, of pathogens and possible sequence variations of their DNA or RNA can be determined using the procedure described. Here, the procedures used for the sequencing or analysis of sequence variations by means of mass spectrometry are known. Reference is made, for example, to the references mentioned at the outset.

Alternatively, it is possible to perform aliquotting of the amplificate or of the sample. The aliquots are then used for primer extension and for base-specific cleavage and correspondingly the concentration and the sequence of the virus DNA is determined.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A procedure for the analysis of a sample, where the sample includes at least one nucleic acid molecule having at least one nucleic acid sequence, the procedure comprising:
making available a comparison molecule of known concentration having a known comparison sequence, where the comparison sequence includes a defined mass difference in comparison to a reference sequence of the nucleic acid sequence;
cleaving the nucleic acid sequence to give fragments of the nucleic acid sequence and the comparison sequence to give fragments of the comparison sequence;
determining a mass spectrum of a mixture of the fragments of the nucleic acid sequence and of the comparison sequence by mass spectrometry;
determining a comparison spectrum of the fragments of the comparison sequence by mass spectrometry;
determination of the concentration of the nucleic acid sequence from the mass spectrum and the comparison spectrum; and
determining still unknown sequence variations of the nucleic acid sequence in comparison to the reference sequence from the mass spectrum and a reference spectrum of the reference sequence.

2. A procedure for the analysis of a sample, where the sample includes at least one nucleic acid molecule having at least one nucleic acid sequence, the procedure comprising:
making available a comparison molecule of known concentration having a known comparison sequence, the comparison sequence having a defined mass difference in comparison to a reference sequence of the nucleic acid sequence;
simultaneously amplifying the nucleic acid molecule and the comparison molecule;
cleaving an amplification product to give fragments of the nucleic acid sequence and to give fragments of the comparison sequence;
determining a mass spectrum of a mixture of the fragments of the nucleic acid sequence and of the comparison sequence by mass spectrometry;
determining a comparison spectrum of the fragments of the comparison sequence by mass spectrometry;
determining the concentration of the nucleic acid sequence from the mass spectrum and the comparison spectrum; and
determining still unknown sequence variations of the nucleic acid sequence in comparison to the reference sequence from the mass spectrum and a reference spectrum of the reference sequence.

3. The procedure as claimed in claim 2, wherein the amplifying is carried out by at least one of cloning, transcription-based amplification, polymerase chain reaction (PCR), ligase chain reaction (LCR) and strand displacement amplification (SDA).

4. The procedure as claimed in claim 1, wherein the comparison sequence is based on the reference sequence corresponding to the nucleic acid sequence and the mass difference is produced by replacing at least one starting base occurring in the reference sequence by at least one labeling base in the reference nucleic acid.

5. The procedure as claimed in claim 1, wherein sequencing of the nucleic acid sequence is carried out by analysis of the molecular weights occurring in the mass spectrum.

6. The procedure as claimed in claim 1, wherein the cleavage of the nucleic acid sequence and the cleavage of the comparison sequence are carried out by addition of at least one restriction enzyme.

7. The procedure as claimed in claim 1, wherein the labeling base and the restriction enzyme are chosen such that the nucleic acid sequence is cleaved at the starting base and the comparison sequence is not cleaved at the labeling base.

8. The procedure as claimed in claim 1, wherein the determination of the concentration of the nucleic acid sequence comprises:
analyzing maxima occurring in the mass spectrum and corresponding to the fragments;
identifying one of the maxima occurring, which corresponds to the fragment of the comparison sequence which comprises the labeling base;
identifying two further maxima which correspond to the two fragments of the nucleic acid sequence which result due to the cleavage of the nucleic acid sequence at the starting base;
determining a concentration ratio of the nucleic acid molecule and of the comparison molecule by determination of the area ratio of one maximum to the sum of the two other maxima; and
determining the concentration of the nucleic acid molecule by division of the concentration ratio by the concentration of the comparison molecule.

9. The procedure as claimed in claim 1, wherein the determination of the sequence variations comprises:
adjusting the comparison spectrum to the concentration of the comparison sequence;
determining a difference spectrum by subtraction of the adjusted comparison spectrum from the mass spectrum; and
determining the sequence variations by comparison of the difference spectrum with the reference spectrum.

10. The procedure as claimed in claim 1, wherein the determination of the mass spectrum and of the comparison spectrum are carried out by at least one of time-of-flight mass spectrometry, matrix-assisted laser desorption/ionization (MALDI), electrospray ionization (ESI), ion-cyclotron resonance (ICR) and a combination of these procedures.

11. The procedure as claimed in claim 1, wherein the determination of the mass spectrum is carried out by at least one of time-of-flight mass spectrometry, matrix-assisted laser desorption/ionization (MALDI), electrospray ionization (ESI), ion-cyclotron resonance (ICR) and a combination of these procedures, and wherein the determination of the comparison spectrum is carried out by a simulation.

12. The procedure as claimed in claim 1, wherein the nucleic acid molecule is DNA.

13. The procedure as claimed in claim 1, wherein the nucleic acid molecule is RNA and before the amplification it is converted into a corresponding cDNA sequence.

14. The procedure as claimed in claim 1, wherein the conversion is carried out by reverse transcriptase.

15. The procedure as claimed in claim 1, wherein the nucleic acid molecule originates from a disease pathogen.

16. The procedure as claimed in claim 1, wherein the nucleic acid molecule is extracted from the sample.

17. The procedure as claimed in claim 1, wherein the extraction of the nucleic acid molecule is carried out by cell disruption with subsequent immobilization of the nucleic acid molecule and washing of the nucleic acid molecule.

18. The procedure as claimed in claim 2, wherein the comparison sequence is based on the reference sequence corresponding to the nucleic acid sequence and the mass difference is produced by replacing at least one starting base occurring in the reference sequence by at least one labeling base in the reference nucleic acid.

19. The procedure as claimed in claim 2, wherein sequencing of the nucleic acid sequence is carried out by analysis of the molecular weights occurring in the mass spectrum.


SEQUENCE LISTING (11 sequences)

SEQ ID NO: 1 - DNA, 17 bp, Artificial
  Only for showing the procedure
actgaagtccggatgga

SEQ ID NO: 2 - DNA, 17 bp, Artificial
  Only for showing the procedure
acggaagtccggatgga

SEQ ID NO: 3 - DNA, 17 bp, Artificial
  Only for showing the procedure
actgaagtccgaatgga

SEQ ID NO: 4 - DNA, 3 bp, Artificial
  Only for showing the procedure
act

SEQ ID NO: 5 - DNA, 5 bp, Artificial
  Only for showing the procedure
gaagt

SEQ ID NO: 6 - DNA, 6 bp, Artificial
  Only for showing the procedure
ccggat

SEQ ID NO: 7 - DNA, 3 bp, Artificial
  Only for showing the procedure
gga

SEQ ID NO: 8 - DNA, 8 bp, Artificial
  Only for showing the procedure
acggaagt

SEQ ID NO: 9 - DNA, 6 bp, Artificial
  Only for showing the procedure
ccggat

SEQ ID NO: 10 - DNA, 3 bp, Artificial
  Only for showing the procedure
gga

SEQ ID NO: 11 - DNA, 6 bp, Artificial
  Only for showing the procedure
ccgaat